(19) United States
(12) Patent Application Publication
(10) Pub. No.: US 2009/0105315 A1
(43) Pub. Date: Apr. 23, 2009

(54) PHENYL-CYCLOALKYL AND PHENYL-HETEROCYCLIC DERIVATIVES AS S1P RECEPTOR AGONISTS

(75) Inventors: Kevin R. Lynch, Charlottesville, VA (US); Timothy L. Macdonald, Charlottesville, VA (US)

Correspondence Address: IPLM GROUP, P.A., POST OFFICE BOX 18455, MINNEAPOLIS, MN, 55418, US

(73) Assignee: University of Virginia Patent Foundation

(21) Appl. No.: 12/195,606

(22) Filed: Aug. 21, 2008

Related U.S. Application Data
(63) Continuation of application No. PCT/US2007/062513, filed on Feb. 21, 2007.
(60) Provisional application No. 60/775,309, filed on Feb. 21, 2006.

(51) Int. Cl. C07D 271/06; A61K 31/4245; A61K 31/381; C07D 333/04; A61P 37/00; A61P 41/00

(52) U.S. Cl. 514/364; 548/131; 514/438; 549/75

(57) ABSTRACT

Compounds that have agonist activity at one or more of the S1P receptors are provided. The compounds are sphingosine analogs that, after phosphorylation, can behave as agonists at S1P receptors.

### CROSS-REFERENCE TO RELATED APPLICATIONS

This application is a continuation of International Application No. PCT/US2007/062513 filed on Feb. 21, 2007, which claims priority under 35 U.S.C. 119 (e) to Provisional Application No. 60/775,309, filed Feb. 21, 2006, the disclosures of which are incorporated by reference in their entirety.

### U.S. GOVERNMENT RIGHTS

This invention was made with United States Government support under Grant No. R01 GM067958, awarded by the National Institutes of Health. The United States Government has certain rights in the invention.

### BACKGROUND

Sphingosine 1-phosphate (S1P) is a lysophospholipid mediator that evokes a variety of cellular responses by stimulation of five members of the endothelial cell differentiation gene (EDG) receptor family. The EDG receptors are G-protein coupled receptors (GPCRs) and, on stimulation, propagate second messenger signals via activation of heterotrimeric G-protein alpha (G_{α}) subunits and beta-gamma (G_{βγ}) dimers. Ultimately, this S1P-driven signaling results in cell survival, increased cell migration and, often, mitogenesis. The recent development of agonists targeting S1P receptors has provided insight regarding the role of this signaling system in physiologic homeostasis. For example, the immunomodulator, FTY720 (2-amino-2-[2-(4-octylphenyl) ethyl] propane 1,3-diol), that, following phosphorylation, is an agonist at 4 of 5 S1P receptors, revealed that enhancing S1P tone influences lymphocyte trafficking. Further, S1P type 1 receptor (S1P₁) antagonists cause leakage of the lung capillary endothelium, which suggests that S1P may be involved in maintaining the integrity of the endothelial barrier in some tissue beds.

Sphingosine 1-phosphate (S1P) is a lysophospholipid mediator that evokes a variety of cellular responses by stimulation of five members of the endothelial cell differentiation gene (EDG) receptor family.

Sphingosine-1-phosphate (S1P) has been demonstrated to induce many cellular processes, including those that result in platelet aggregation, cell proliferation, cell morphology, tumor-cell invasion, endothelial cell chemotaxis and angiogenesis. For these reasons, S1P receptors are good targets for therapeutic applications such as wound healing and tumor growth inhibition.

Sphingosine-1-phosphate signals cells in part via a set of G protein-coupled receptors named S1P₁, S1P₂, S1P₃, S1P₄, and S1P₅ (formerly EDG1, EDG5, EDG3, EDG6 and EDG8). The EDG receptors are G-protein coupled receptors (GPCRs) and on stimulation propagate second messenger signals via activation of heterotrimeric G-protein alpha (G_{α}) subunits and beta-gamma (G_{βγ}) dimers. These receptors share 50-55% amino acid sequence identity and cluster with three other receptors (LPA₁, LPA₂, and LPA₃ (formerly EDG2, EDG4 and EDG7) for the structurally related lysophosphatidic acid (LPA).

A conformational shift is induced in the G-Protein Coupled Receptor (GPCR) when the ligand binds to that receptor, causing GDP to be replaced by GTP on the α-subunit of the associated G-proteins and subsequent release of the G-proteins into the cytoplasm. The α-subunit then dissociates from the βγ-subunit and each subunit can then associate with effector proteins, which activate second messengers leading to a cellular response. Eventually, the GTP on the G-proteins is hydrolyzed to GDP and the subunits of the G-proteins reassociate with each other and then with the receptor. Amplification plays a major role in the general GPCR pathway. The binding of one ligand to one receptor leads to the activation of many G-proteins, each capable of associating with many effector proteins leading to an amplified cellular response.

S1P receptors make good drug targets because individual receptors are both tissue and response specific. Tissue specificity of the S1P receptors is desirable because development of an agonist or antagonist selective for one receptor localizes the cellular response to tissues containing that receptor, limiting unwanted side effects. Response specificity of the S1P receptors is also of importance because it allows for the development of agonists or antagonists that initiate or suppress certain cellular responses without affecting other responses. For example, the response specificity of the S1P receptors could allow for an S1P mimetic that initiates platelet aggregation without affecting cell morphology.

Sphingosine-1 -phosphate is formed as a metabolite of sphingosine in its reaction with sphingosine kinase and is stored in platelets where high levels of sphingosine kinase exist and S1P lyase is lacking. S1P is released during platelet aggregation, accumulates in serum, and is also found in malignant ascites. Reversible biodegradation of S1P most likely proceeds via hydrolysis by ectophosphohydrolases, specifically the sphingosine 1-phosphate phosphohydrolases. Irreversible degradation of S1P is catalyzed by S1P lyase yielding ethanolamine phosphate and hexadecenal.

Currently, there is a need for potent and selective agents that are agonists of the S1P receptor. There is also a need for pharmacological tools for the further study of the physiological processes associated with agonism of the S1P receptors.

### SUMMARY

The present invention provides, in one aspect, sphingosine-1-phosphate analogs that are potent and selective agonists at one or more S1P receptors, specifically the S1P₁ receptor type. In another aspect, the compounds can have a phosphate moiety as well as a hydrolysis-resistant phosphate surrogates such as phosphonate, alpha-substituted phosphonate (particularly where the alpha-substitution is a halogen), and phosphothionates. In addition, the invention provides pro-drugs, such as, primary alcohol containing compounds that can be activated or converted, (e.g., phosphorylated) in vitro, e.g., by sphingosine kinase enzyme, most particularly sphingosine kinase type 2 (SPHK2).

The present invention provides in one aspect sphingosine-1-phosphate analogs having formula I or formula II:

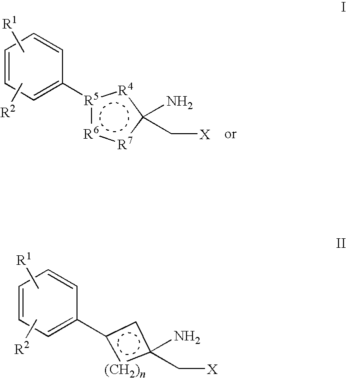

wherein R⁴ and R⁷ are independently CH, or CH₂; R⁵ is C, CH, or N, R⁶ is CH, CH₂, O, S or NR³; R³ is hydrogen, or (C₁-C₁₀)alkyl; X is hydroxyl (—OH), phosphate (—OPO₃H₂), phosphonate (—CH₂PO₃H₂), or alpha-substituted phosphonate; R¹ is hydrogen, halo (C₁-C₁₀)alkyl, or (C₁-C₁₀)alkoxy; R² is a group having formula III, IV, V, or VI:

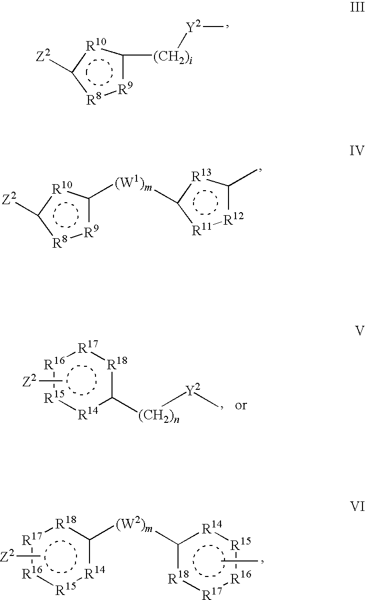

wherein R⁸,R⁹, R¹⁰, R¹¹, R¹², R¹³, R¹⁴, R¹⁵, R¹⁶, R¹⁷ and R¹⁸ are independently O, S, C, CR¹⁹, CR²⁰R²¹, C═O, N or NR²²; R¹⁹, R²⁰ and R²¹ are independently hydrogen, halo, (C₁-C₁₀)alkyl, (C₁-C₁₀)alkyl substituted with halo, hydroxy, (C₁-C₁₀)alkoxy, or cyano; R²² is hydrogen or (C₁-C₁₀)alkyl; and at least one ring of the formula III, IV, V, or VI groups includes a heteroatom (O, S or N); Z² is (C₁-C₆)alkyl, (C₃-C₈)cycloalkyl, (C₂-C₆)alkenyl, (C₂-C₆)alkynyl, (C₆-C₁₀)aryl, (C₇-C₁₆)alkaryl, or (C₇-C₁₆)arylalkyl; wherein the alkyl groups of Z² are optionally substituted with 1, 2, 3, or 4 substituent groups, where the substituent groups independently are halo, (C₁-C₁₀)alkoxy or cyano; [symbol] indicates one or more optional double bonds; Y² is a bond, —O—, or >C═O; W¹ and W² are —CH₂—, where m is 0, 1, 2 or 3; or W² is —(C═O)(CH₂)₁₋₅—, where m is 1; n is 0, 1, 2, 3 or 4; i is 0, 1, 2, 3 or 4; and q is 0, 1, 2, or 3.

The alkyl groups of R¹ can be optionally substituted with 1, 2, 3, or 4 substituent groups, where the substituent groups independently are aryl, (C₁-C₁₀)alkoxy or cyano. Any of the alkyl, alkenyl, alkynyl, cycloalkyl, aryl, heterocyclic, or heteroaryl groups of R² are optionally substituted with 1, 2, 3, or 4 substituent groups, where the substituent groups independently are oxo (═O), imino (═NR^{d}), (C₁-C₁₀)alkyl, (C₁-C₁₀)alkoxy, or C₆-aryl, or wherein one or more of the carbon atoms in the R² alkyl groups can be independently replaced with non-peroxide oxygen, sulfur or NR^{c}; the alkyl groups of R³ are optionally substituted with 1, or 2 hydroxy groups; and R^{c} and R^{d} are independently hydrogen, or (C₁-c₁₀)alkyl. The invention includes pharmaceutically acceptable salts or esters of the compounds of formula I or formula II.

In another aspect, the present invention also provides esters of any of the compounds of formula I or formula II, e.g., phosphate esters or phosphonate esters.

In another aspect, the invention provides compounds of formula I or formula II that are phosphate esters, having formula VII.

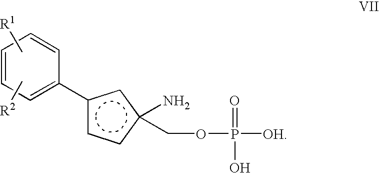

In another aspect, the invention provides pro-drugs of the compounds of formula I or formula II. In another aspect, the invention also provides compounds of formula I or formula II for use in medical therapy.

In another aspect, the present invention provides a method for inhibiting angiogenesis in a tumor, comprising contacting the cancerous cells with an effective amount of a compound of formula I or formula II.

In another aspect, the invention provides a method for modulating the immune system by altering lymphocyte trafficking for treatment of autoimmune diseases or prolongation of allograft transplant survival, said method comprising administering an effective amount of at least one compound of formula I or formula II to a subject in need thereof.

In another aspect, the invention provides a method for preventing, inhibiting or treating neuropathic pain, wherein the method comprises administering an effective amount of at least one compound of formula I, formula II or a compound of formula I or formula II with a pharmaceutically-acceptable carrier to a subject in need thereof. Pain can be nociceptive or neuropathic in nature. Neuropathic pain is characterized by its chronic nature, an absence of an obvious, direct cause (e.g., tissue damage), hyperalgesia or allodynia. Hyperalgesia is an exaggerated response to a painful stimulus. Allodynia is the perception of normal stimuli as painful (examples include the touch of clothing, warm or cool air, etc.). Neuropathic pain can be a sequel to nerve damage in an extremity such as an arm, or more often a leg. Precipitating events can include trauma, e.g., motor vehicle accidents or amputations (e.g., phantom limb pain). Neuropathic pain can occur due to an adverse effect of drug therapies, e.g., vincristine or paclitaxel (TAXOL™) or can occur as a component of disease pathologies, such as diabetes type 1 or type2, shingles, HIV-1 infections, etc. Typically, neuropathic pain is not responsive to opiates or non-steroidal anti-inflammatory drugs such as aspirin.

In another aspect, the invention provides a method for repairing vascular injury following catheterization, comprising contacting the lumen of the affected vessel with an effective amount of the compound of formula I or formula II. In another aspect, the invention includes coating indwelling stents with a compound of formula I or formula II.

In another aspect, the present invention provides compositions and methods for the use of S1P analogs to prevent and inhibit vascular restenosis following vascular injury. For example, the injury can be due to balloon angioplasty. In another aspect, the present invention includes a method for treating subjects to prevent vascular restenosis.

In another aspect, the present invention provides compositions and methods for the use of sphingosine analogs (including S1P pro-drugs) to prevent asthma attacks. In one aspect, the asthma could be due to over production of cysteinyl leukotrienes. In another aspect, the present invention includes a method for treating asthma.

In another aspect, the present invention provides compositions and methods for the use of sphingosine analogs of formula I or formula II (including S1P pro-drugs) to treat obesity.

In another aspect, the present invention provides compositions and methods for the use of sphingosine analogs (including S1P pro-drugs) to normalize blood lipid composition. In one aspect, blood low density lipoprotein (LDL or ‘bad cholesterol’) levels could be lowered. In another aspect, blood triglyceride levels may be lowered by administration of an effective amount of a compound having formula I or formula II.

In another aspect, the present invention provides compositions and methods for the use of S1P analogs and S1P pro-drugs for the prevention and treatment of arteriosclerosis.

In another aspect, the present invention provides compositions and methods for the use of S1P analogs and S1P pro-drugs for the treatment of neoplastic disease. In one aspect, this treatment is effected by application of S1P receptor antagonists having formula I or formula II that are efficacious by virtue of their anti-angiogenic properties. In another aspect, the treatment is effected by administration of sphingosine analogs of formula I or formula II that inhibit the multiple substrate lipid kinase.

In another aspect, the present invention provides compositions and methods for the use of S1P analogs and S1P pro-drugs for the treatment of neurodegenerative diseases. In one aspect, the treatment is for senile dementia of the Alzheimers type.

In another aspect, the invention provides a compound of formula I or formula II, or a pharmaceutically acceptable salt thereof for use in medical treatment (for example, treatment of neoplastic disease, treatment of neuropathic pain, treatment of autoimmune disease, prolongation of allograft survival).

In another aspect, the invention provides for the use of a compound of formula I or formula II to prepare a medicament for inhibiting tumor growth, metastasis or tumor angiogenesis in a mammalian species (for example, a human).

In another aspect, the invention provides for the use of a compound of formula I or formula II to prepare a medicament for treating an autoimmune disease or prolonging allograft survival in a mammalian species (for example, a human).

In another aspect, the invention provides for the use of a compound of formula I or formula II to prepare a medicament for treating neuropathic pain in a mammalian species (for example, a human).

In another aspect, the invention provides a method for assessing a compound of formula I or formula II (e.g., S1P receptor pro-drugs) as a substrate for sphingosine kinase types 1 or 2, in vitro and in vivo. In another aspect, the invention includes a method of assessing a compound of formula I or formula II for binding to designated receptor sites comprising in vivo or in vitro, with an amount of a compound of formula I or formula II effective to bind said receptors. Tissue comprising ligand bound designated S1P receptor sites can be used to measure the selectivity of test compounds for specific receptor subtypes, or can be used as a tool to identify potential therapeutic agents for the treatment of diseases, by contacting said agents with said ligand-receptor complexes, and measuring the extent of displacement of the ligand or binding of the agent.

In another aspect, the invention provides novel intermediates and processes disclosed herein that are useful for preparing compounds of formula I or formula II, including the generic and specific intermediates as well as the synthetic processes described herein.

In another aspect, the present invention provides synthetic schemes and methods of use of compounds having formula I, formula II, analogs or derivatives thereof. In another aspect, the invention provides synthetic and modification schemes for preparing analogs and derivatives of the compounds of formula I or formula II, as well as compositions and methods for the use of such analogs and derivatives.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

The details of one or more embodiments of the invention are set forth in the accompanying description below. Other features, objects, and advantages of the invention will be apparent from the description and drawings, and from the claims.

### BRIEF DESCRIPTION OF THE DRAWINGS

FIGS. 1-3 illustrate the syntheses of the disclosed compounds.

### DETAILED DESCRIPTION

The following abbreviations are used herein: S1P, sphingosine-1-phosphate; S1P₁₋₅ S1P receptor types; GPCR, G-protein coupled receptor; SAR, structure-activity relationship; EDG, endothelial cell differentiation gene; EAE, experimental autoimmune encephalomyelitis; NOD non-obese diabetic; TNFα, tumor necrosis factor alpha; HDL, high density lipoprotein; and RT-PCR, reverse transcriptase polymerase chain reaction.

In describing and claiming the invention, unless otherwise defined, all technical and scientific terms used herein have the same meaning as commonly understood by one of ordinary skill in the art to which this invention belongs. Although any materials and methods similar or equivalent to those described herein can be used in the practice or testing of the present invention, the preferred materials and methods are described herein. Each of the following terms has the meaning associated with it in this section. Specific and preferred values listed below for radicals, substituents, and ranges are for illustrations only; they do not exclude other defined values or other values within defined ranges for the radicals and substituents.

The terms “a,” “an,” “the,” “at least one,” and “one or more” are used interchangeably. Thus, for example, a composition that comprises “an” element means one element or more than one element.

The term “receptor agonists” are compounds that mimic the action of S1P at one or more of its receptors but may have differing potency or efficacy.

The term “receptor antagonists” are compounds that 1) lack intrinsic agonist activity and 2) block agonist (e.g., S1P) activation of the S1P receptor(s), often in a manner that is both fully surmountable and reversible (‘competitive antagonist’).

The term “affected cell” refers to a cell of a subject afflicted with a disease or disorder, which affected cell has an altered phenotype relative to a subject not afflicted with a disease or disorder.

Cells or tissue are “affected” by a disease or disorder if the cells or tissue have an altered phenotype relative to the same cells or tissue in a subject not afflicted with a disease or disorder.

A disease or disorder is “alleviated” if the severity of a symptom of the disease or disorder, the frequency with which such a symptom is experienced by a patient, or both, is reduced.

An “analog” of a chemical compound is a compound that, by way of example, resembles another in structure but is not necessarily an isomer (e.g., 5-fluorouracil is an analog of thymine).

The terms “cell,” “cell line,” and “cell culture” may be used interchangeably.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one that, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

A “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” means an amount sufficient to produce a selected effect. For example, an effective amount of an S1P receptor antagonist is an amount that decreases the cell signaling activity of the S1P receptor.

A “functional” molecule is a molecule in a form in which it exhibits a property by which it is characterized. By way of example, a functional enzyme is one that exhibits the characteristic catalytic activity by which the enzyme is characterized.

The term “inhibit” refers to the ability of a disclosed compound to reduce or impede a described function. Preferably, inhibition is by at least 10%, more preferably by at least 25%, even more preferably by at least 50%, and most preferably, the function is inhibited by at least 75%.

“Instructional material” includes a publication, a recording, a diagram, or any other medium of expression that can be used to communicate the usefulness of the disclosed compounds in the kit for effecting alleviation of the various diseases or disorders recited herein. Optionally, or alternately, the instructional material may describe one or more methods of alleviating the diseases or disorders in a cell or a tissue of a mammal. The instructional material of the kit may, for example, be affixed to a container that contains a disclosed compound or be shipped together with a container that contains the identified compound. Alternatively, the instructional material may be shipped separately from the container with the intention that the instructional material and the compound be used cooperatively by the recipient.

The term “parenteral” means not through the alimentary canal but by some other route such as subcutaneous, intramuscular, intrathecal, or intravenous.

The term “pharmaceutically acceptable carrier” includes any of the standard pharmaceutical carriers, such as a phosphate buffered saline solution, water and emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

The term “purified” and similar terms relate to the isolation of a molecule or compound in a form that is substantially free (at least 75% free, preferably 90% free, and most preferably at least 95% free) from other components normally associated with the molecule or compound in a native environment. The term “purified” does not necessarily indicate that complete purity of the particular molecules achieved during the process. A “very pure” compound refers to a compound that is greater than 90% pure. A “highly purified” compound refers to a compound that is greater than 95% pure.

A “sample” refers preferably to a biological sample from a subject, including, but not limited to, normal tissue samples, diseased tissue samples, biopsies, blood, saliva, feces, semen, tears, and urine. A sample can also be any other source of material obtained from a subject, which contains cells, tissues, or fluid of interest. A sample can also be obtained from cell or tissue culture.

The term “standard,” refers to something used for comparison. For example, a standard can be a known standard agent or compound that is administered or added to a control sample and used for comparing results when measuring said compound in a test sample. Standard can also refer to an “internal standard,” such as an agent or compound that is added at known amounts to a sample and is useful in determining such things as purification or recovery rates when a sample is processed or subjected to purification or extraction procedures before a marker of interest is measured.

A “subject” of analysis, diagnosis, or treatment is an animal. Such animals include mammals, preferably a human.

A “therapeutic” treatment is a treatment administered to a subject who exhibits signs of pathology for the purpose of diminishing or eliminating those signs.

A “therapeutically effective amount” of a compound is that amount of compound that is sufficient to provide a beneficial effect to the subject to which the compound is administered.

The term “treating” includes prophylaxis of the specific disorder or condition, or alleviation of the symptoms associated with a specific disorder or condition or preventing or eliminating said symptoms.

The disclosed compounds are generally named according to the IUPAC or CAS nomenclature system. Abbreviations that are well known to one of ordinary skill in the art may be used (e.g., “Ph” for phenyl, “Me” for methyl, “Et” for ethyl, “h” for hour or hours, “rt” for room temperature, and “rac” for racemic mixture).

The values listed below for radicals, substituents, and ranges, are for illustration only; they do not exclude other defined values or other values within defined ranges for the radicals and substituents. The disclosed compounds include compounds of formula I or formula II having any combination of the values, specific values, more specific values, and preferred values described herein.

The term “halogen” or “halo” includes bromo, chloro, fluoro, and iodo. The term “haloalkyl”, refers to an alkyl radical bearing at least one halogen substituent, non-limiting examples include, but are not limited to, chloromethyl, fluoroethyl or trifluoromethyl and the like. The term “(C₁-C₁₀)alkyl” refers to a branched or linear alkyl group having from one to ten carbons. Non-limiting examples include, but are not limited to, methyl, ethyl, n-propyl, iso-propyl, butyl, iso-butyl, sec-butyl, tert-butyl, pentyl, hexyl, heptyl, octyl and the like. The term “(C₂-C₆)alkenyl”, refers to an olefinically unsaturated branched or linear group having from two to six carbon atoms and at least one double bond. Typically, (C₂-C₆)alkenyl groups include, but are not limited to, 1-propenyl, 2-propenyl, 1,3-butadienyl, 1-butenyl, hexenyl, pentenyl, hexenyl, and the like. The term (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1- hexynyl, 2-hexynyl, 3-hexynyl, 4-hexynyl, or 5-hexynyl, and the like. The carbon atoms of the alkenyl or alkynyl groups that are not multiply bonded are considered alkyl carbon atoms for purposes of substitution or replacement. The term “(C₁-C₁₀)alkoxy” refers to an alkyl group attached through an oxygen atom. Examples of (C₁-C₁₀)alkoxy can be methoxy, ethoxy, propoxy, isopropoxy, butoxy, iso-butoxy, sec-butoxy, pentoxy, 3-pentoxy, or hexyloxy and the like. The term “(C₃-C₈)cycloalkyl”, can be cyclopropyl, cyclobutyl, cyclopentyl, cyclohexyl, cycloheptyl, cyclooctyl and the like.

The term “optionally substituted” refers to zero, one, two, three or four substituents, wherein the substituents are each independently selected. Each of the independently selected substituents may be the same or different than other substituents.

The term “(C₆-C₁₀)aryl” refers to a mono or bicyclic carbocyclic ring system having one or two aromatic rings including, but not limited to, phenyl, benzyl, naphthyl, tetrahydronaphthyl, indanyl, indenyl, and the like.

The term “(C₇-C₁₆)arylalkyl” or “(C₇-C₁₆)aralkyl” refers to an alkyl group substituted with a mono or bicyclic carbocyclic ring system having one or two aromatic rings including, a group such as phenyl, naphthyl, tetrahydronaphthyl, indanyl, indenyl, and the like. Non-limiting examples of arylalkyl include benzyl, phenylethyl, and the like.

The term “optionally substituted aryl” includes aryl compounds having zero, one, two, three or four substituents, and a substituted aryl includes aryl compounds having one, two, three or four substituents, wherein the substituents include groups such as, for example, alkyl, halo, or amino substituents.

The “(C₂-C₁₀)heterocyclic group” refers to an optionally substituted mono- or bicyclic carbocyclic ring system containing one, two, or three heteroatoms (optionally in each ring) wherein the heteroatoms are oxygen, sulfur, and nitrogen.

The term “(C₄-C₁₀)heteroaryl” refers to an optionally substituted mono- or bicyclic carbocyclic ring system containing one, two, or three heteroatoms (optionally in each ring) wherein the heteroatoms are oxygen, sulfur, and nitrogen. Non-limiting examples of heteroaryl groups include furyl, thienyl, pyridyl, and the like.

The term “phosphate analog” and “phosphonate analog” comprise analogs of phosphate and phosphonate wherein the phosphorous atom is in the +5 oxidation state and one or more of the oxygen atoms is replaced with a non-oxygen moiety, including for example, the phosphate analogs phosphorothioate, phosphorodithioate, phosphoroselenoate, phosphorodiselenoate, phosphoroanilothioate, phosphoranilidate, phosphoramidate, boronophosphates, and the like, including associated counterions, e.g., H, NH₄, Na, K, and the like if such counterions are present.

A “derivative” of a compound refers to a chemical compound that may be produced from another compound of similar structure in one or more steps, such as replacement of hydrogen by an alkyl, acyl, or amino group.

The term “pharmaceutically acceptable carrier” includes any of the standard pharmaceutical carriers, such as a phosphate buffered saline solution, hydroxypropyl beta-cyclodextrins (HO-propyl beta cyclodextrins), water, emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

The term “pharmaceutically-acceptable salt” refers to salts that retain the biological effectiveness and properties of the disclosed compounds and which are not biologically or otherwise undesirable. In many cases, the disclosed compounds are capable of forming acid or base salts by virtue of the presence of amino or carboxyl groups or groups similar thereto.

An “effective amount” means an amount sufficient to produce a selected effect. For example, an effective amount of an S1P receptor agonist is an amount that decreases the cell signaling activity of the S1P receptor.

The disclosed compounds can contain one or more asymmetric centers in the molecule. In accordance with the present disclosure any structure that does not designate the stereochemistry is to be understood as embracing all the various optical isomers, as well as racemic mixtures thereof.

The disclosed compounds may exist in tautomeric forms and the invention includes both mixtures and separate individual tautomers. For example, the following structure:

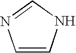

is understood to represent a mixture of the structures:

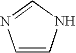

as well as

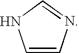

The terms 16:0, 18:0, 18:1, 20:4 or 22:6 hydrocarbon refers to a branched or straight alkyl or alkenyl group, wherein the first integer represents the total number of carbons in the group and the second integer represent the number of double bonds in the group.

An “S1P modulating agent” refers a compound or composition that is capable of inducing a detectable change in S1P receptor activity in vivo or in vitro (e.g., at least 10% increase or decrease in S1P activity as measured by a given assay such as the bioassay described in the examples and known in the art. “S1P receptor,” refers to all of the S1P receptor subtypes (for example, the S1P receptors S1P₁, S1P₂, S1P₃, S1P₄, and S1P₅), unless the specific subtype is indicated.

It will be appreciated by those skilled in the art that the disclosed compounds having chiral centers may exist in and be isolated in optically active and racemic forms. It is to be understood that the disclosed compounds encompass any racemic, optically active or stereoisomeric form, or mixtures thereof, of the compound, which possess the useful properties described herein, such as the S,R; S,S; R,R; or R,S diastereomers. It is well known in the art how to prepare such optically active forms (for example, by resolution of the racemic form by recrystallization techniques, by synthesis from optically-active starting materials, by chiral synthesis, or by chromatographic separation using a chiral stationary phase) and how to determine S1P agonist activity using the standard tests described herein, or using other similar tests that are well known in the art. In addition, some compounds may exhibit polymorphism.

Potential uses of an S1P receptor agonist pro-drugs (S1P₁ receptor type selective agonists preferred) include, but are not limited to, altering lymphocyte trafficking as a method of treatment for autoimmune pathologies such as uveitis, type I diabetes, rheumatoid arthritis, inflammatory bowel diseases, and, most particularly, multiple sclerosis. “Treatment” of multiple sclerosis includes the various forms of the disease including relapsing-remitting, chronic progressive, etc., and the S1P receptor agonists can be used alone or in conjunction with other agents to relieve signs and symptoms of the disease as well as prophylactically.

In addition, the disclosed compounds can be used for altering lymphocyte trafficking as a method for prolonging allograft survival, for example solid organ transplants, treatment of graft vs. host disease, bone marrow transplantation, and the like.

In addition, the disclosed compounds can be used to inhibit autotaxin. Autotaxin, a plasma phosphodiesterase, has been demonstrated to undergo end product inhibition. Autotaxin hydrolyzes several substrates to yield lysophosphatidic acid and sphingosine 1-phosphate, and has been implicated in cancer progression and angiogenesis. Therefore, S1P receptor agonist pro-drugs of the disclosed compounds can be used to inhibit autotaxin. This activity may be combined with agonism at S1P receptors or may be independent of such activity.

In addition, disclosed compounds can be useful for inhibition of S1P lyase. S1P lyase is an intracellular enzyme that irreversibly degrades S1P. Inhibition of S1P lyase disrupts lymphocyte trafficking with concomitant lymphopenia. Accordingly, S1P lyase inhibitors can be useful in modulating immune system function. Therefore, the disclosed compounds can be used to inhibit S1P lyase. This inhibition could be in concert with S1P receptor activity, or be independent of activity at any S1P receptor.

In addition, disclosed compounds can be useful as antagonists of the cannabinoid CB₁ receptor. CB₁ antagonism is associated with a decrease in body weight and an improvement in blood lipid profiles. The CB₁ antagonism could be in concert with S1P receptor activity, or be independent of activity at any S1P receptor.

In addition, disclosed compounds can be useful for inhibition of group IVA cytosolic PLA₂ (cPLA₂). cPLA₂ catalyzes the release of eicosanoic acids (e.g., arachidonic acid). The eicosanoic acids are transformed to pro-inflammatory eicosanoids such as prostaglandins and leukotrienes. Thus, disclosed compounds may be useful as anti-inflammatory agents. This inhibition could be in concert with S1P receptor activity, or be independent of activity at any S1P receptor.

In addition, disclosed compounds may be useful for inhibition of the multiple substrate lipid kinase (MuLK). MuLK is highly expressed in many human tumor cells and thus its inhibition might slow the growth or spread of tumors.

“Treatment” of multiple sclerosis includes the various forms of the disease including relapsing-remitting, chronic progressive, etc., and the S1P receptor agonists can be used alone or in conjunction with other agents to relieve signs and symptoms of the disease as well as prophylactically.

The present invention is also includes pharmaceutical compositions comprising the compounds of formula I or formula II. More particularly, such compounds can be formulated as pharmaceutical compositions using standard pharmaceutically acceptable carriers, fillers, solubilizing agents and stabilizers known to those skilled in the art. For example, a pharmaceutical composition comprising a compound of formula I or formula II, or analog, derivative, or modification thereof, as described herein, is used to administer the appropriate compound to a subject.

The compounds of formula I or formula II are useful for treating a disease or disorder including administering to a subject in need thereof of a therapeutically acceptable amount of a compound of formula I or formula II, or a pharmaceutical composition comprising a therapeutically effective amount of a compound of formula I or formula II, and a pharmaceutically-acceptable carrier.

The disclosed compounds and method are directed to sphingosine 1-phosphate (S1P) analogs that have activity as receptor agonists or antagonists at one or more S1P receptors, specifically the S1P₁, S1P₄ and S1P₅ receptor types. The disclosed compounds and method include both compounds that have a phosphate moiety as well as compounds with hydrolysis-resistant phosphate surrogates such as phosphonates, alpha-substituted phosphonates particularly where the alpha substitution is a halogen and phosphothionates.

The values listed below for radicals, substituents, and ranges, are for illustration only; they do not exclude other defined values or other values within defined ranges for the radicals and substituents.

A preferred value for n is 0, 1, 2, or 3.

A preferred value for R⁶ is CH, CH₂, O, N or NH.

A preferred value for R⁴, R⁵, R⁶ and R⁷ are CH or CH₂.

A preferred value for lower alkyl group is methyl, ethyl or propyl.

A preferred value for halo is fluorine or chlorine.

A preferred value for X is hydroxy or OPO₃H₂.

Alpha-substituted phosphonate includes —CHFPO₃H₂, —CF₂PO₃H₂, —CHOHPO₃H₂, —C═OPO₃H₂ or thiophosphate (OPO₂SH₂).

A preferred value for R¹ is hydrogen.

Preferred cyclic groups including a double bond include:

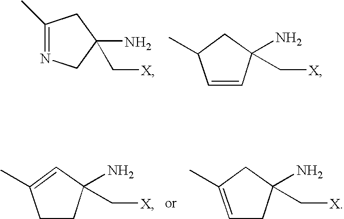

A preferred compound of the invention has the R¹ group placed ortho or meta to R².

Additional preferred compounds have the R group placed para to the cyclic group (e.g., 1,4).

Non-limiting examples of esters of the compounds include compounds where the X group is;

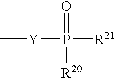

wherein Y is O, CH₂, CHOH, CHF, CF₂, or

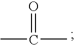

and R²⁰ and R²¹ are alkoxy, alkenyloxy, alkynyloxy, aryloxy,

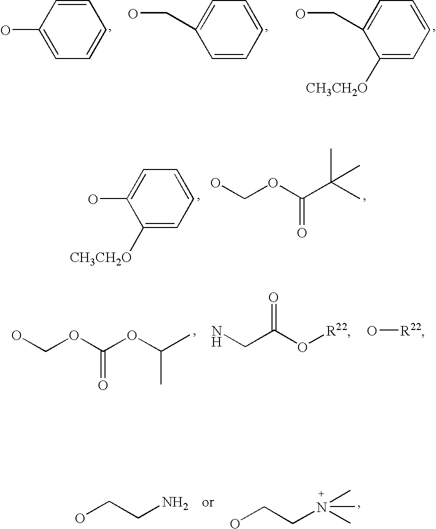

wherein R²² is C₁-C₄ alkyl, C₂-C₄ alkenyl, C₂-C₄ alkynyl, or optionally substituted aryl. Preferred R²⁰ and R²¹ groups are alkoxy,

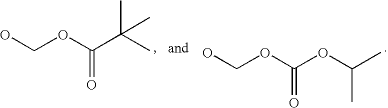

Preferred compounds of formula I include:

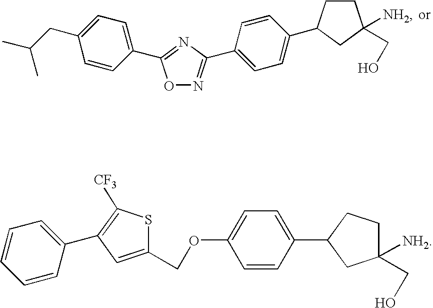

Additional preferred compounds of formula I include:

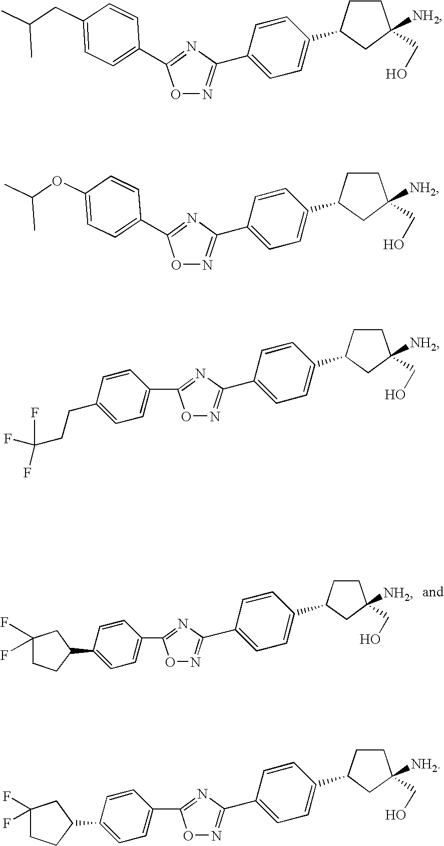

Additional compounds of formula I are illustrated in table 1, below.

TABLE 1

| | |
|---|---|
| | A |
| 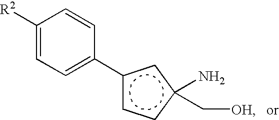 | |
| | B |
| 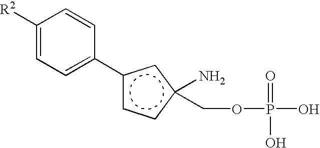 | |

| Compound | R² |
|---|---|
| VIII | 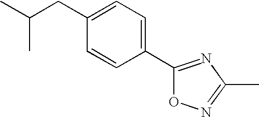 |
| IX | 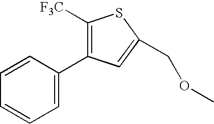 |
| X | 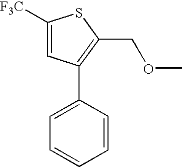 |
| XI | 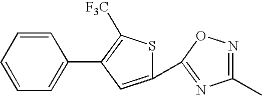 |
| XII | 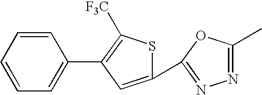 |
| XIII | 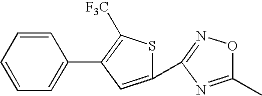 |

Without wishing to be bound by any particular theory, it is expected that the compounds described herein are pro-drugs, e.g., are activated by phosphorylation of the primary alcohol to form the mono-phosphorylated analog. Additionally, the active drugs are expected to be agonists at the S1P type 1 receptor.

In cases where compounds of formula I are sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases, include but are not limited to, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are alkyl, substituted alkyl, alkenyl, substituted alkenyl, cycloalkyl, substituted cycloalkyl, cycloalkenyl, substituted cycloalkenyl, aryl, heteroaryl, or heterocyclic and the like. Also included are amines where the two or three substituents, together with the amino nitrogen, form a heterocyclic or heteroaryl group. Non-limiting examples of amines include, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful, for example, carboxylic acid amides, including carboxamides, lower alkyl carboxamides, dialkyl carboxamides, and the like.

The compounds of formula I can be formulated as pharmaceutical compositions and administered to a mammal, such as a human patient in a variety of forms adapted to the chosen route of administration, e.g., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Exemplary pharmaceutical dosage forms for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, e.g., when they are liquids. However, it will generally be desirable to administer them to the skin as compositions or formulations, in combination with a dermatologically acceptable carrier, which may be a solid or a liquid.

Exemplary solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of formula I in a liquid composition, such as a lotion, will be from about 0.1 to about 25 weight percent, preferably from about 0.5-10 weight percent. The concentration in a semi-solid or solid composition such as a gel or a powder will be about 0.1-5 wt-%, preferably about 0.5-2.5 weight percent based on the total weight of the composition.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a dose will be in the range of from about 0.1 to about 10 mg/kg of body weight per day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 1.0 to about 1000 nanoM, preferably, about 10 to 500 nanoM, most preferably, about 25 to about 200 nanoM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four, or more sub-doses per day. The sub-dose itself may be further divided, e.g., into a number of discrete loosely spaced administrations; such as multiple inhalations from an insufflator or by application of a plurality of drops into the eye.

The disclosed method includes a kit comprising an inhibitor compound of formula I and instructional material that describes administering the inhibitor compound or a composition comprising the inhibitor compound to a cell or a subject. This should be construed to include other embodiments of kits that are known to those skilled in the art, such as a kit comprising a (preferably sterile) solvent for dissolving or suspending the inhibitor compound or composition prior to administering the compound or composition to a cell or a subject. Preferably, the subject is a human.

In accordance with the disclosed compounds and methods, as described above or as discussed in the Examples below, there can be employed conventional chemical, cellular, histochemical, biochemical, molecular biology, microbiology, and in vivo techniques that are known to those of skill in the art. Such techniques are explained fully in the literature.

Without further description, it is believed that one of ordinary skill in the art can, using the preceding description and the following illustrative examples, make and utilize the disclosed compounds.

Processes for preparing compounds of formula I or for preparing intermediates useful for preparing compounds of formula I are provided as further embodiments. Intermediates useful for preparing compounds of formula I are also provided as further embodiments. The processes are provided as further embodiments and are illustrated in the schemes, wherein the meanings of the generic radicals are as given above unless otherwise qualified.

The invention is now described with reference to the following Examples and Embodiments. Without further description, it is believed that one of ordinary skill in the art can, using the preceding description and the following illustrative examples, make and utilize the disclosed compounds. The following working examples therefore, are provided for the purpose of illustration only and specifically point out the preferred embodiments, and are not to be construed as limiting in any way the remainder of the disclosure. Therefore, the examples should be construed to encompass any and all variations that become evident as a result of the teaching of the specification.

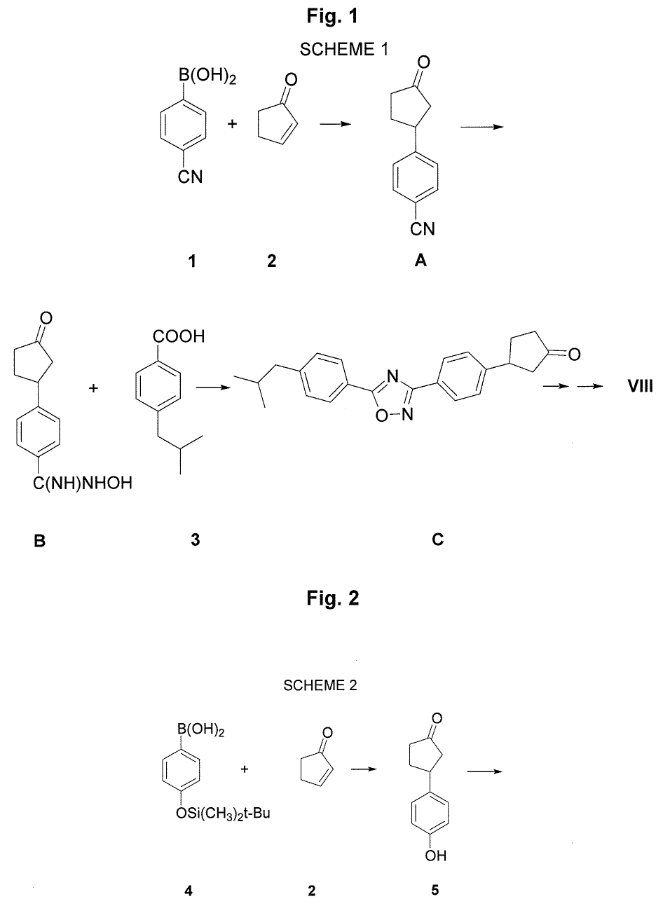

The disclosed compounds presented above in Table 1 can be synthesized by the routes illustrated in either Scheme 1 (FIG. 1) or Scheme 2 (FIG. 2). In Scheme, 1 the key steps in the synthesis involve initial coupling of a 4-cyanophenyl boronic acid, 1, with cyclopentenone, 2, and subsequent conversion of the nitrile of compound A, to form compound C. Compound C can be converted to compound VIII, shown in Scheme 3.

In a similar manner, the cyclopentanone intermediate to XI can be prepared. Additional alterations in this sequence could produce precursors for intermediates to XII and XIII. Using the phenol derived from the modifications to the synthetic scheme noted in Example 2, below, the appropriate intermediate cyclopentanone compounds for IX and X can be synthesized.

In Scheme 2 the key steps involve preparation of a phenolic cyclopentanone using 4-tertbutyldimethylsilyloxyphenyl boronic acid. After generation of the desired cyclopentanone intermediate, the carbonyl function is elaborated into the 1-amino-1-hydroxymethyl unit as described below.

### EXAMPLES

### Example 1

### 3-(4′-Cyanophenyl)cyclopentanone (Compound A)

Palladium (II) acetate (0.23 g, 0.1 eq) and antimony (III) chloride (0.23 g, 0.1 eq) are added to a solution of 80 mL acetic acid containing 2-cyclopenten-1-one, 2, (0.82 g, 10 mmol), sodium acetate (1.6 g, 20 mmol) and 4-cyanophenyl boronic acid, 1, (1.46 g, 10 mmol) under nitrogen. The reaction is stirred for 24 hours at 25° C., the black precipitate is filtered off and the filtrate diluted with 250 mL of brine, extracted twice with 50 mL of methylene chloride. The organic layer is stirred with saturated bicarbonate solution for 30 minutes, washed with brine and dried over magnesium sulfate. The solvent is removed and chromatographed to provide compound A.

### Example 2

### 3-(4′-hydroxylphenyl)cyclopentanone.

Palladium (II) acetate (0.23 g, 0.1 eq) and antimony (III) chloride (0.23 g, 0.1 eq) are added to a solution of 80 mL acetic acid containing 2-cyclopenten-1-one, 2, (0.82 g, 10 mmol), sodium acetate (1.6 g, 20 mmol) and 4-tertbutyldimethyylsilyloxyphenyl boronic acid, 4, (2.54 g, 10 mmol) under nitrogen. The reaction is stirred for 24 hours at 25° C., the black precipitate is filtered off and the filtrate diluted with 250 mL of brine, then extracted twice with 50 mL of methylene chloride. The organic layer is stirred with saturated bicarbonate solution for 30 minutes, washed with brine and dried over magnesium sulfate. The solvent is removed and chromatographed to provide 3-(4′-hydroxyphenyl)-cyclopentanone.

### Example 3

### 3-(4′-Aldoximinophenyl)cyclopentanone (Compound B)

Compound A (1.0 mmol) is dissolved in 95% ethanol (1.5 mL). Triethylamine (2.3 mmol) and hydroxylamine hydrochloride (2.2 mmol) are added and the reaction mixture is heated to about 75° C. for 3 hours. The reaction progress can be monitored by TLC. Generally, after about 3 hours, no starting nitrile remains and the solution is concentrated to a slurry and from water, or an organic solvent. The solid is filtered and washed with cold water, and vacuum dried to provide the crude product, which can be used in the next step without further purification.

### Example 4

### 3-{4-[5-(4-Isobutyl-phenyl)-[1,2,4]oxadiazol-3-yl]-phenyl}-cyclopentanone (Compound C)

To a solution of 4-isobutylbenzoic acid, 3, (0.150 mmol) in dry methylene chloride (4 mL) are added (benzotriazol-1-yloxy)tripyrrolidinophosphonium hexaflurophosphate (PyBOP) (0.150 mmol) and diisopropyl ethylamine (0.150 mmol), followed by the aldoximinophenyl derivative (compound B) (0.150 mmol). The reaction is stirred at room temperature for about 12-16 hours. The mixture is diluted with diethyl ether (15 mL), washed with saturated aqueous ammonium chloride (2×5 mL), brine (5 mL), and concentrated in vacuo. The title compound is purified by column chromatography.

### Example 5

### Conversion of the 3-(4′-Substituted phenyl)cyclopentanone Intermediates into Compounds VIII-XIII

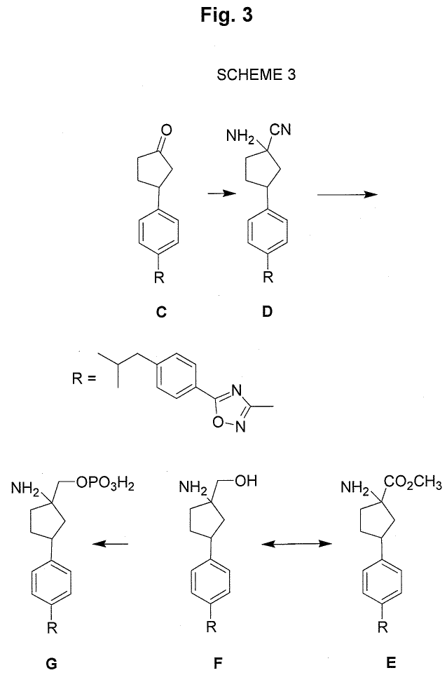

The cyclopentanone intermediates synthesized through the sequences outlined in Scheme 1 can be converted into the 1-amino-1-hydroxymethyl-3-(4′-substituted phenyl)cyclopentanes (compounds VIII-XIII) through a 3 step procedure described in International Patent Application WO 2006/088944 A1, pages 37-39. This procedure is illustrated for the synthesis of compound VIII in FIG. 3. The cyclopentaone precursors to IX-XIII can be converted through analogous methods.

### Step 1

### 1-Amino-3-(4′-substituted phenyl)cyclopentanecarbonitrile (Scheme 3: Compound D)

The cyclopentanone intermediate (11.8 mmol), sodium cyanide (0.15 g, 23.5 mmol) and ammonium chloride (1.25 g, 23.5 mmol) are added to 20 mL of aqueous ammonium hydroxide solution. The mixture is vigorously stirred overnight. After completion the reaction mixture is extracted twice with 10 mL of methylene chloride after. The organic extraction is dried over magnesium sulphate, concentrated to provide the amino nitrile, D. The crude product is used for the next step without further purification. (See e.g., J. Med. Chem., 1986, 29, 1988-1995.)

In a similar manner the cyclopentanone intermediate to XI can be prepared. Alterations in this sequence can produce the precursors for intermediates to XII and XIII. Using the phenol derived from the modifications to the synthetic scheme noted above, the appropriate intermediate cyclopentanone compounds for IX and X can be synthesized.

### Step 2

### 1-Amino-3-(4′-substituted phenyl)cyclopentanecarboxylic acid (Scheme 3; Compound E)

The crude product from step 1 (11.2 mmol) and 50 mL concentrated hydrochloric acid are heated to about 70° C. and stirred overnight under argon or nitrogen atmosphere. The resulting aqueous solution is evaporated to dryness. Water 10 mL is added and the solution is dried again. This process is repeated twice. The crude product is washed with cold water and acetone to provide compound E.

### Step 3

### [1-Amino-3-(4′-phenyl)cyclopentanyl]methanol (Scheme 3; Compound F)

The product from step 2 (0.20 mmol) and sodium borohydride (27 mg, 0.6 mmol) is dissolved in 3 mL of tetrahydrofuran. After the solution is cooled to about 0° C., 51 mg (0.2 mmol) of iodine dissolved in 1 mL THF is added dropwise. The vessel is fitted with a condenser and the reaction mixture is heated at reflux under argon for 5 hours. The excess sodium borohydride is quenched with methanol. After solvent removal by evaporation in vacuo, 2 mL water and 5 mL methylene chloride are added and the mixture is stirred for about 1 hour. The organic phase is collected and the aqueous phase is extracted twice with methylene chloride. The combined organic extracts are dried and concentrated to provide the crude product. Further purification by column chromatography provides the purified compound.

### Step 4

### Conversion to the Phosphate (Scheme 3: Compound G)

The alcohols, VIII-XIII can be converted into the corresponding phosphates by the following procedure. 1 mL 85% aqueous phosphoric acid is slowly added to 0.5 g of phosphorus pentoxide, heated at 100° C. for 1 hour under nitrogen. Another 0.5 g of phosphorus pentoxide and 30 mg of the alcohol VIII (or IX-XIII) are added to the mixture and the reaction heated for an additional 5 hours. After cooling to room temperature, 10 mL ice water is added to the reaction mixture. The product is collected as a precipitate. The product is collected and washed with water, then dried under vacuum.

The assays below are standard literature reported assays known in the art for confirming and quantifying the activity of the disclosed compounds.

### Example 6

### Sphingosine Kinase Assay

Recombinant sphingosine kinase type 2 (SPHK2) is prepared by forcing the expression of the mouse or human recombinant enzyme by transfecting the relevant plasmid DNA into HEK293T or CHO K1 cells. After about 60 hours, cells are harvested, broken and the non-microsomal (e.g., soluble) fraction is retained. The broken cell supernatant fluid containing the recombinant enzyme is mixed with test compounds (FTY720, AA151, VIII and XVIII) (5-50 micromolar) and γ-32P-ATP and incubated for 0.5-2.0 hours at 37° C. The lipids in the reaction mixture are extracted into an organic solvent and displayed by normal phase thin layer chromatography. The radio-labeled bands are detected by autoradiography, scraped from the plate and quantified by scintillation counting. The test compounds are used at a concentration of about 50 μM, incubation time is about 20 minutes.

### Example 7

### GTPγS-35 Binding Assay

This assay illustrates agonist activation of G protein coupled receptors (GPCRs) in isolation. The assay forces expression concomitantly of a recombinant GPCR (e.g., the S1P1-5 receptor) and each of the three subunits (typically, α-i2, β-1, and γ-2) of a heterotrimeric G protein in a HEK293T cell by transfecting the cell with four plasmid DNAs encoding the respective proteins. About 60 hours after transfection the cells are harvested, broken, the nucleus discarded, and the crude microsomes are prepared from the remainder. Agonist (e.g., S1P) stimulation of the receptor-G protein complex on the microsomes results in the exchange of GTP for GDP on the α-subunit in a dose-dependent manner. The GTP-bound α-subunit is detected using a GTP analog (GTPγS-35), which is a radionuclide (sulfur-35) labeled phosphothionate that is not hydrolyzed to GDP. The microsomes with the adherent G proteins are collected by filtration and the bound GTPγS-35 quantified in a liquid scintillation counter. The assay yields relative potency (EC₅₀ values) and maximum effect (efficacy, Eₘₐₓ). Antagonist activity is detected as rightward shifts in the agonist dose-response curve in the presence of a fixed amount of antagonist. If the antagonist behaves competitively, the affinity of the receptor/antagonist pair (Kᵢ) can be determined. The assay is described in Davis, M. D., J. J. Clemens, T. L. Macdonald and K. R. Lynch (2005) “S1P Analogs as Receptor Antagonists” Journal of Biological Chemistry, vol. 280, pp. 9833-9841.

### Example 8

### Lymphopenia Assay

Compounds (e.g., primary alcohols test compounds) are dissolved in 2% hydroxypropyl beta-cyclodextrin and introduced into groups of mice by oral gavage at doses from .01, 1.0 and 10 mg/kg body weight. At intervals, e.g., 24 hours, 48 hours, or 96 hours the mice are lightly anesthetized and ca. 0.1 mL of blood is drawn from the orbital sinus. The number of lymphocytes (in thousands per microliter of blood; normal is 4-11) is determined using a Hemavet blood analyzer.

### Example 9

### Heart Rate Assay

Mice are dosed with test compounds (intravenous, 3 mg/kg) or vehicle (2% hydroxypropyl beta-cyclodextrin) and the heart rate measured at 1 hour post dosing. Heart rate is captured in unrestrained, conscious animals using the ECGenie™ system.

The invention should not be construed to be limited solely to the assays and methods described above, but should be construed to include other methods and assays as well. Other methods that are used but not described above are well known and within the competence of one of ordinary skill in the art of chemistry, biochemistry, molecular biology, and clinical medicine. One of ordinary skill in the art will know that other assays and methods are available to perform the procedures described above.

The abbreviations used above have their conventional meaning within the clinical, chemical, and biological arts. In the case of any inconsistencies, the present disclosure, including any definitions therein will prevail.

The disclosures of each and every patent, patent application, and publication cited in the specification are expressly incorporated herein by reference in their entirety into this disclosure. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this specification and the claims shown below are not limited to the illustrative embodiments set forth.

We claim:

1. A compound of formula I or formula II: 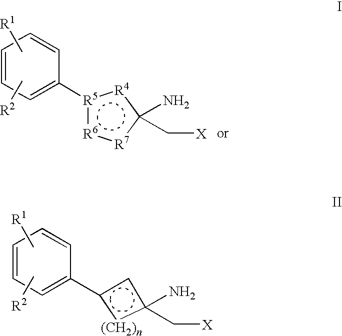
wherein R⁴and R⁷ are independently CH, or CH₂; R⁵ is C, CH, or N, R⁶is CH, CH₂, O, S or NR³; R³ is hydrogen or (C₁-C₁₀)alkyl; X is hydroxyl (—OH), phosphate (—OPO₃H₂), phosphonate (—CH₂PO₃H₂), or alpha-substituted phosphonate;
R¹ is hydrogen, halo, (C₁-C₁₀)alkyl, (C₁-C₁₀)haloalkyl, or (C₁-C₁₀)alkoxy;
R² is a group having formula III, IV, V or VI: 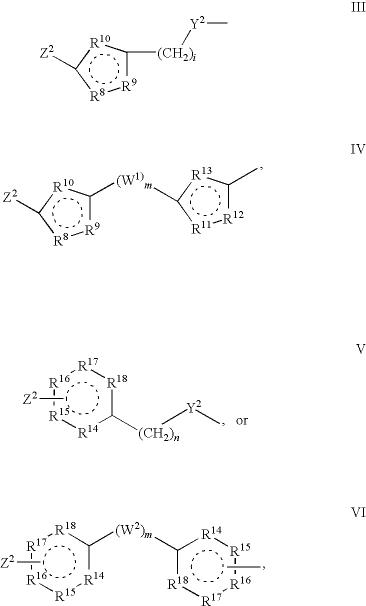
wherein R⁸,R⁹, R¹⁰, R¹¹, R¹², R¹³, R¹⁴, R¹⁵, R¹⁶, R¹⁷ and R¹⁸ are independently O, S, C, CR¹⁹, CR²⁰OR²¹, C═O, N or NR ; R¹⁹, R²⁰ and R²¹ are independently hydrogen, halo, (C₁-C₁₀)alkyl, (C₁-C₁₀)alkyl substituted with halo, hydroxy, (C₁-C₁₀)alkoxy, or cyano; R²² is hydrogen or (C₁-C₁₀)alkyl; and at least one ring of the formula III, IV, V, or VI groups includes a heteroatom (O, S or N); Z iS (C₁-C₆)alkyl, (C₃-C₈)cycloalkyl, (C₂-C₆)alkenyl, (C₂-C₆)alkynyl, (C₆-CIO)aryl, (C₇-C₁₆)alkaryl, or (C₇-C₁₆)arylalkyl; wherein the alkyl groups of Z are optionally substituted with 1, 2, 3, or 4 substituent groups, where the substituent groups independently are halo, (C₁-C₁₀)alkoxy or cyano; [symbol] indicates one or more optional double bonds; Y² is a bond, —O—, or >C═O; W¹ and W² are —CH₂—, where m is 0, 1, 2 or 3; or W² is —(C═O)(CH₂)₁₋₅—, where m is 1; n is 0, 1, 2, 3 or 4; i is 0, 1, 2, 3 or 4; and q is 0, 1, 2, or 3.
wherein the alkyl groups of R¹ can be optionally substituted with 1, 2, 3, or 4 substituent groups, where the substituent groups independently are aryl, (C₁-C₁₀)alkoxy or cyano; and the alkyl, alkenyl, alkynyl, cycloalkyl, aryl, heterocyclic, or heteroaryl groups of R² are optionally substituted with 1, 2, 3, or 4 substituent groups, where the substituent groups independently are oxo (═O), imino (═NR^{d}), (C₁-C₁₀)alkyl, (C₁-C₁₀)alkoxy, or C₆-aryl, or wherein one or more of the carbon atoms in the R² alkyl groups can be independently replaced with non-peroxide oxygen, sulfur or NR^{c}; the alkyl groups of R³ are optionally substituted with 1, or 2 hydroxy groups; and R^{d} is hydrogen, or (C₁-C₁₀)alkyl; or a pharmaceutically acceptable salt or ester thereof.

2. The compound of claim 1, wherein R¹ is hydrogen, fluorine, chlorine, bromine, trifluoromethyl, methoxy, (C₁-C₆)alkyl, (C₁-C₆)haloalkyl, or (C₁-C₆)alkyl substituted with, alkoxy, cyano or aryl.

3. The compound of claim 2, wherein R¹ is hydrogen, trifluoromethyl, or —CH₂CF₃.

4. The compound of claim 2, wherein R¹ is benzyl, phenylethyl, or methyl benzyl.

5. The compound of claim 1, wherein R² is

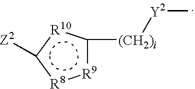

6. The compound of claim 5, wherein R² is: 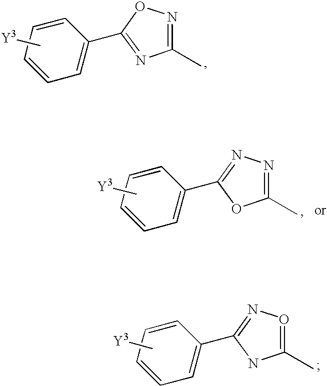
where Y³ is (CH₃)₃C—, CH₃CH₂(CH₃)₂C—, CH₃CH₂CH₂—, CH₃(CH₂)₂CH₂—, CH₃(CH₂)₄CH₂—, (CH₃)₂CHCH₂—, (CH₃)₃CCH₂—, CH₃CH₂O—, (CH₃)₂CHO—, or CF₃CH₂CH₂— or a group having the formula:

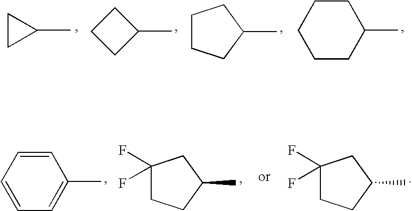

7. The compound of claim 6, wherein R² is:

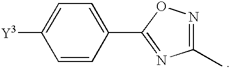

8. The compound of claim 7, wherein R² is:

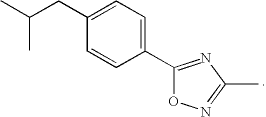

9. The compound of claim 5, wherein R² is:

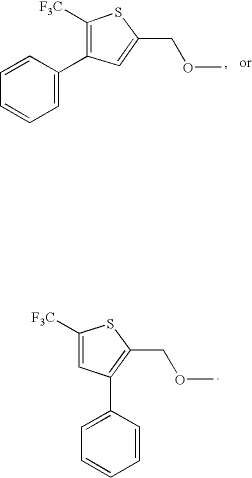

10. The compound of claim 9, wherein R² is

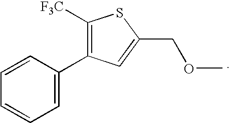

11. The compound of claims 1, wherein R² has formula IV

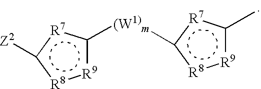

12. The compound of claim 11, wherein R² is

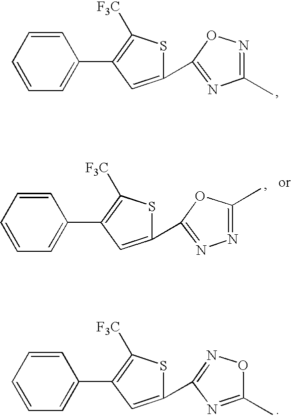

13. The compound of claim 1, wherein each of X¹, Y¹ and Z¹ is C or CH₂.

14. The compound of claim 1, wherein R³ is hydrogen, methyl, hydroxymethyl, ethyl, hydroxyethyl, propyl, or isopropyl.

15. The compound of claim 14, wherein R³ is hydrogen, methyl, hydroxymethyl, ethyl, or hydroxyethyl.

16. The compound of claim 1, having the formula:

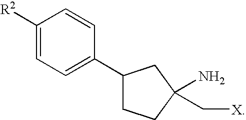

17. The compound of claim 16, having the formula

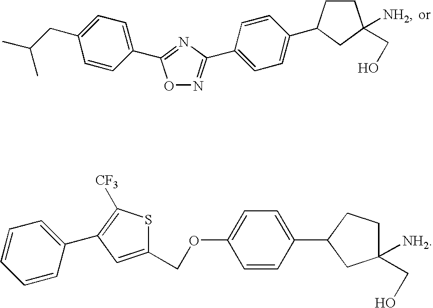

18. A method for prevention or treatment of a pathological condition or symptom in a mammal, wherein the activity of sphingosine 1-phosphate receptors is implicated and agonism of such activity is desired, comprising administering to said mammal an effective amount of a compound of claim 1.

19. The method of claim 18, wherein the pathological condition is an autoimmune disease.

20. The method of claim 19, wherein the autoimmune disease is uveitis, type I diabetes, rheumatoid arthritis, inflammatory bowel diseases, or multiple sclerosis.

21. The method of claim 20, wherein the autoimmune disease is multiple sclerosis.

22. The method of claim 18, wherein prevention or treatment of the pathological pathological condition is altering lymphocyte trafficking.

23. The method of claim 23, wherein altering lymphocyte trafficking provides prolonged allograft survival.

24. The method of claim 24 wherein the allograft is for transplantation.

25. A method for prevention or treatment of a pathological condition or symptom in a mammal, wherein the activity S1P lyase implicated and inhibition of the S1P lyase is desired, comprising administering to said mammal an effective amount of a compound of claim 1.
